(19) United States
(12) Patent Application Publication
(10) Pub. No.: US 2010/0240608 A1
(43) Pub. Date: Sep. 23, 2010

(54) PARTICLE STABILISED OIL-IN-WATER EMULSION

(75) Inventor: Nicholas David Hedges, Sharnbrook (GB)

Correspondence Address: UNILEVER PATENT GROUP, 800 SYLVAN AVENUE, AG West S. Wing, ENGLEWOOD CLIFFS, NJ, 07632-3100, US

(73) Assignee: CONOPCO, INC., D/B/A UNILEVER, Englewood Cliffs, NJ (US)

(21) Appl. No.: 12/726,750

(22) Filed: Mar. 18, 2010

(30) Foreign Application Priority Data
Mar. 20, 2009 (EP) EP09155790

(51) Int. Cl. A61K 31/715; A61K 31/732; A61K 31/729; A61P 43/00; A23D 7/00

(52) U.S. Cl. 514/61; 514/54; 426/602

(57) ABSTRACT

The present invention relates to an oil-in-water emulsion comprising a gelled particle emulsifier derived from naturally occurring food-grade polymers, the emulsion preferably being in the form of a food product or a home care product or a personal care product or a pharmaceutical product.

Emulsifiers are limited in their use as they can cause allergic reactions in some people. There is thus a constant need for alternative emulsifiers. The goal of the present invention is to provide a stable emulsion which can be used in a wide number of applications.

Thus an oil-in-water emulsion comprising 0.001-50%, preferably 0.001-30%, more preferably 0.001-10% w/w oil and 0.001 to less than 0.5%, preferably 0.001-0.4%, more preferably 0.01-0.4% w/w a gelled particle emulsifier, wherein the gelled particle emulsifier comprises at least one gellable polysaccharide, wherein the gelled particle emulsifier has a largest dimension of 3-1000 nm, preferably 5-500 nm, more preferably 10-200 nm, and wherein the emulsion composition does not comprise further emulsifier.

The present invention relates to an oil-in-water emulsion composition comprising a gelled particle emulsifier derived from naturally occurring food-grade polymers, the emulsion composition preferably being in the form of a food product or a home care product or a personal care product or a pharmaceutical product.

Emulsifiers are limited in their use as they can cause allergic reactions in some people. There is thus a constant need for alternative emulsifiers.

Pickering emulsions advantages and characteristics are well known and US2002/0054890 describes Pickering emulsions wherein amphiphilic particles are first dispersed in an aqueous phase and then the aqueous phase is combined with the oil phase. In this case, no gel particles are formed, indeed no gel at all is formed since the aqueous phase is combined with the oil phase. The amphiphilic particles can be modified polysaccharides (by etherification, esterification or selective oxidation), their average diameter of the modified polysaccharides particles is less 20 microns, preferably less than 15 microns.

The fact that such modified polysaccharides are used to stabilise emulsions leads to quite large quantities having to be used (0.1% to 30% w/w of the total emulsion).

It has now been found that, by selecting certain polysaccharides, it is possible to form gel particles of the size required in a Pickering emulsion and to use said gel particles to stabilise emulsions.

The term “food-grade” means compounds, which are safe for use in food as defined by governmental institutes such as the FDA in the US or the WHO. In Europe food grade may be defined as an ingredient that has an E number. For example, agar is E406, gellan is E418 and pectins are E440.

### SUMMARY OF THE INVENTION

Thus the invention provides an oil-in-water emulsion comprising 0.001-50%, preferably 0.001-30%, more preferably 0.001-10% w/w oil and 0.001 to less than 0.5%, preferably 0.001-0.4%, more preferably 0.01-0.4% w/w a gelled particle emulsifier, wherein the gelled particle emulsifier comprises at least one gellable hydrophilic polysaccharide, wherein particles of the gelled particle emulsifier have a largest dimension of 3-1000 nm, preferably 5-500 nm, more preferably 10-200 nm.

When describing the polysaccharide as hydrophilic, it is meant that it is not only hydrophilic but that it is not amphiphilic.

By the term “largest dimension” is meant the longest straight dimension that can be measured, in this case, on the particle. The largest dimension of the gelled particle emulsifier can be determined by any commonly known processes such as electron microscopy or light scattering methods. Typically the ratio of the largest dimension of the droplets which form the dispersed phase of the emulsion to the largest dimension of the gelled particle emulsifier is in the range 10:1-10000:1, preferably 10:1-1000:1. It has been observed that the superior emulsion stability provided by the gelled particle emulsifier of the invention allows stabilisation of larger droplets up to a largest dimension of about 100 microns.

The gelled particle emulsifier has, when in use, a water content of 30-99.95%, typically 90-95% w/w based on the total weight of the gelled particle emulsifier. Thus the gelled particle emulsifier is softer and less abrasive and therefore the inventive emulsion compositions may be used in applications where harder abrasive particles would not be preferred. It also means than less emulsifier is actually required than in the prior art. Preferably, the emulsion according to the invention contains less than 0.1%, more preferably less than 0.05%, even more preferably less than 0.01% hydrophilic polysaccharide.

It has been noted that polysaccharides, most of which are naturally occurring food-grade polymers, are normally used to form open and extensive gel networks for use as, for example, thickeners and gelling agents, rather than gelled particles. Thus the polysaccharides must undergo processing in order to form a gelled particle emulsifier as detailed herein below.

Preferably the weight ratio of gelled particle emulsifier to oil is 0.001-0.5:1, preferably 0.005-0.25:1, more preferably 0.01-0.1:1. Thus, quite low levels of gelled particle emulsifier may be used to stabilise quite large levels of oil in the form of oil-in-water emulsions. In fact, due to the large levels of water in the gelled particle emulsifiers, the actual amount of gellable polysaccharide used to provide emulsion stability is very low.

The gelled particle emulsifier can have an aspect ratio in the range of 1:1 to 10:1: Preferably the gelled particle emulsifier is in the form of a sphere, a disk or a rod.

The gellable polysaccharide may be selected from the group consisting of agar, agarose, gellan pectin alginates and caggageenans. Preferred gellable polysaccharides are neutral or weakly charged polymers such as agar or agarose although charged polymers may be used depending on the pH and ionic strength.

Optionally the emulsion comprises at least one further emulsifier and/or further food or non-food ingredients.

The gelled particle emulsifier can be formed by any method that provides particles of the correct size and surface properties. The method will typically begin with forming a solution of polymer followed by one or more process steps including chemical treatment or enzymatic treatment of the polymer and cooling, mixing, drying, freezing and concentrating. In one method a hot solution of polymer is prepared and the polymer chains hydrolysed such that on cooling a suspension of particles rather than a gel forms. Hydrolysis may be carried out using any suitable method such as acid, alkaline or enzymatic hydrolysis.

The emulsion may be in the form of a food product or a home care product or a personal care product or a pharmaceutical product. It has been observed that particle stabilised emulsions are very stable, it is thought, because the particles do not appear to migrate from the oil-water interface and thus are particularly useful when employed to stabilise emulsions where there are a plurality of oil-water interfaces.

### SUMMARY OF THE FIGURES

The invention is now illustrated with reference to the following figures which show in:

FIG. 1 the particle size distribution of the agar gelled particle emulsifier of example 1 obtained by light scattering;

FIG. 2 the particle size distribution of the pectin gelled particle emulsifier of example 2 obtained by light scattering;

FIG. 3 the oil droplet size distribution of dodecane-in-water emulsions stabilised by 0.5% w/w agar gelled particle emulsifier according to example 4 obtained by light scattering for 2%, and 10% w/w dodecane;

FIG. 4 the oil droplet size distribution of dodecane-in-water emulsions stabilised by 0.25% w/w agar gelled particle emulsifier according to example 5 obtained by light scattering for 2%, 6% and 10% w/w dodecane;

FIG. 5 the oil droplet size distribution of dodecane-in-water emulsions stabilised by 0.1% w/w agar gelled particle emulsifier according to example 6 obtained by light scattering for 1%, 2%, 6% and 10% w/w dodecane;

FIG. 6a the variation of D(4,3) and D(3,2) with agar gelled particle emulsifier to dodecane weight ratio for the dodecane-in-water emulsions of examples 4, 5 and 6;

FIG. 6b the variation of D(4,3) and D(3,2) with total amount of agar to dodecane weight ratio for the dodecane-in-water emulsions of examples 4, 5 and 6;

FIG. 7 the oil droplet size distribution of silicon oil-in-water emulsions stabilised by 0.5% w/w agar gelled particle emulsifier according to example 7 obtained by light scattering for 10%, 20% and 30% w/w silicon oil.

FIG. 8 the oil droplet size distribution of silicon oil-in-water emulsions stabilised by 0.5% w/w agar gelled particle emulsifier according to example 7 obtained by light scattering for 10%, 20% and 30% w/w silicon oil after 2 weeks storage at chill temperature;

FIG. 9 the oil droplet size distribution of MCT oil-in-water emulsions stabilised by 0.5% w/w agar gelled particle emulsifier according to example 8 obtained by light scattering for 10% w/w and 20% w/w MCT oil;

FIG. 10 the oil droplet size distribution of silicon oil-in-water emulsions stabilised by 0.5% w/w agar gelled particle emulsifier according to example 8 obtained by light scattering for 10% and 20% w/w MCT oil after 2 weeks storage at chill temperature;

FIG. 11 the oil droplet size distribution of soya bean oil oil-in-water emulsions stabilised by 0.25% w/w agar gelled particle emulsifier according to example 9 obtained by light scattering for 10% soya bean oil emulsions;

FIG. 12 the oil droplet size distribution of soya bean oil oil-in-water emulsions stabilised by 0.25% w/w agar gelled particle emulsifier according to example 9 obtained by light scattering for 10% w/w soya bean oil after 1 weeks storage at chill temperature;

FIG. 13 the oil droplet size distribution of dodecane-in-water emulsions stabilised by 0.5% w/w pectin gelled particle emulsifier according to example 2 obtained by light scattering for 2% and 10% w/w dodecane;

FIG. 14 the oil droplet size distribution of dodecane-in-water emulsions stabilised by 0.25% w/w pectin gelled particle emulsifier according to example 2 obtained by light scattering for 2%, 6% and 10% w/w dodecane;

FIG. 15 the oil droplet size distribution of the dodecane-in-water emulsions of FIG. 8 after 1 week of storage at chill temperature;

FIG. 16 the oil droplet size distribution of dodecane-in-water emulsions stabilised by 0.1% w/w pectin gelled particle emulsifier according to example 2 obtained by light scattering for 1%, 2%, 6% and 10% w/w dodecane;

FIG. 17 the oil droplet size distribution of dodecane-in-water emulsions stabilised by 0.5% w/w pectin gelled particle emulsifier according to example 2 obtained by light scattering for 10%, 15%, 20%, 30%, 40% and 50% w/w dodecane;

FIG. 18a the variation of D(4,3) and D(3,2) with pectin gelled particle emulsifier to dodecane weight ratio for the dodecane-in-water emulsions of examples 10 and 11.

FIG. 18b the variation of D(4,3) and D(3,2) with amount of pectin to dodecane weight ratio for the dodecane-in-water emulsions of examples 10 and 11.

### DETAILED DESCRIPTION OF THE INVENTION

### Example 1

### Preparation of Agar Gelled Particle Emulsifier

Deionised water was heated to above 95° C. and kept covered in order to avoid evaporation. 1% w/w of agar powder (Luxara agar (code: 1254) from Arthur Branwell Ltd) was added slowly and allowed to dissolve fully and heated for at least 30 minutes or until all the agar had dissolved. The resulting solution was allowed to cool to 70° C. 1% w/w citric acid was then added and the solution held at 70° C. for 4 hours. The solution was then cooled to chill temperature (around 5° C.) and stored at chill temperature until required.

The hydrolysate was allowed to sediment overnight and then the upper clear layer decanted off. The lower layer was shaken to mix well and then transferred into a 50 ml centrifuge tubes. The hydrolysate was collected by centrifugation for 10 minutes at 3000 revolutions per minute (rpm) on a bench centrifuge (MSE Centaur 2 centrifuge). The resulting pellet of hydrolysate was re-suspended in approximately five times its volume of deionised water and centrifuged once more. This process was repeated a further two times. After the final centrifugation, the liquid (supernatant) was decanted off leaving the final hydrolysate. This particulate material was then used to prepare emulsions. The hydrolysate was stored at chill temperature in a weak solution of citric acid (e.g. 0.025M or 0.05M), until used.

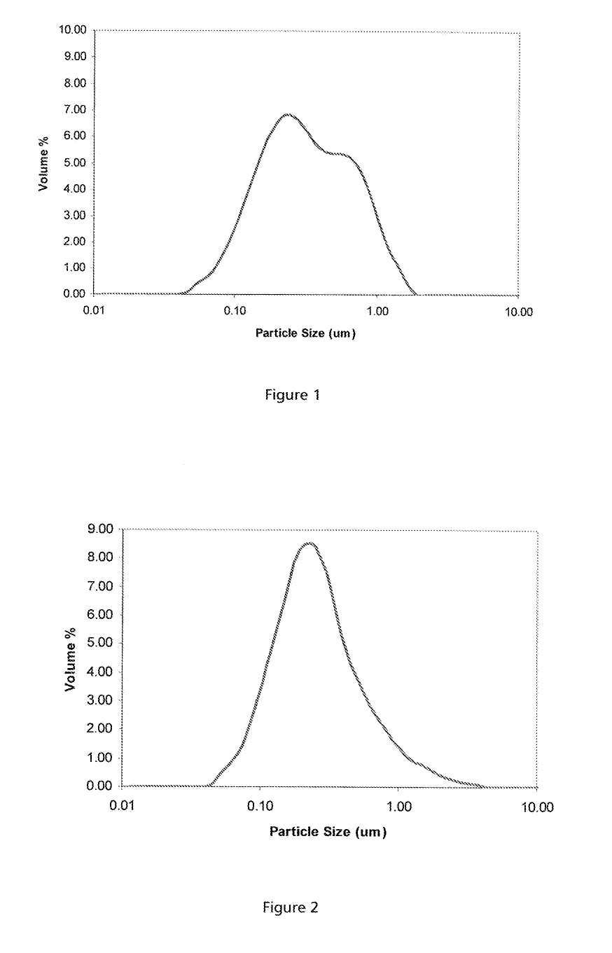

The particle size was determined using a Malvem Mastersizer 2000 fitted with a small volume sample dispersion unit. Samples were measured in deionised water at room temperature. The particulate material was dispersed in distilled water using a Silverson LR4 mixer running at full speed for 3 minutes in order to re-disperse the particulate material fully. The refractive index of the dispersing medium was 1.33, the refractive index of the dispersed particles was 1.335, the size range was 0.02 μm to 2000 μm and the analysis model was the General Purpose Spherical Model. The particle size was recorded after the final washing and re-dispersion step. The results are shown in FIG. 1. It should be noted that the resolution limit of the Malvern Mastersizer is around 20 nm, so that particles below this size are not resolved. The volume weighted particle size D(4,3) and the mean surface area weighted or Sauter mean particle size D(3,2). were 0.38 μm and 0.22 μm respectively

The water content of the particles, determined from an analysis of the dry weight was 92% w/w. These particles are used in examples 3, 4, 5 and 6

### Example 2

### Preparation of Pectin Gelled Particle Emulsifier

2% w/w pectin powder (Sigma Pectin, esterified potassium salt from citrus fruit Code P9311) was mixed with water at 100° C. and stirred for 1 hour to fully dissolve. The solution was then cooled to 70° C. and the pH adjusted by the addition of citric acid to 3.0. The resulting solution was gently stirred for 2 hours. The pH was then adjusted to 1.0 using HCl and the mixture stirred for a further 15 minutes at 70° C. Finally the solution was combined with an equal volume of 1% w/w aqueous CaCl₂ to yield an aqueous solution of 1% w/w pectin and 0.5% w/w CaCl₂.

This solution was cooled to chill temperature (4° C.) and particles allowed to form. The particles were then washed thrice with 0.5% w/w aqueous CaCl₂ solution using the protocol described in example 1, with the final wash solution also containing 0.1% w/w citric acid and 0.1% w/w potassium sorbate in water.

The particle size was determined by the method described in example 1. FIG. 2 shows the particle size distribution of the pectin particles. The volume weighted particle size D(4,3) and the mean surface area weighted or Sauter mean particle size D(3,2). were 0.34 μm and 0.19 μm respectively. The water content of the particles, determined from an analysis of the dry weight, was 93% w/w.

### Example 3

### Preparation of Dodecane-in-Water Emulsions Stabilised with 0.5% or 0.25% W/W Agar Gelled Particle Emulsifier

The gelled particle emulsifier of example 1 was collected from the storage solution by centrifugation for 10 minutes at 3000 rpm in a bench centrifuge (MSE Centaur 2 centrifuge). The supernatant was discarded and 0.5% or 0.25% w/w gelled particle emulsifier was re-dispersed in water using a Silverson LR4 high shear mixer set at high speed for 3 minutes. To this dispersion was added either 2%, 6% or 10% w/w dodecane (Sigma-Aldrich code: 022,110-4). Emulsions were then prepared using a Silverson L4R high shear mixer set at high speed (speed setting 5) for 2 minutes.

Table 1 shows the oil droplet size date measured on a Malvern Mastersizer 2000. The method was the same as that used in example 1, except the refractive index of the dispersed particles (dodecane) was set to 1.421. The droplet sizes quoted are the mean volume weighted drop size D(4,3) and the mean surface area weighted or Sauter mean drop size, D(3,2). Also shown in table 1 are the oil droplet size data after 3 weeks storage at 5° C. Inspection of the data in table 1 suggests little increase in oil droplet size with storage time (NB: the particle concentrations given in parentheses are the particle concentrations in the aqueous phase).

TABLE 1
Agar gelled particle emulsifier stabilised emulsion oil droplet
size data for the dodecane-in-water emulsions of example 3,

| Agar | | | | | |
|---|---|---|---|---|---|
| gelled particle | | D(4, 3) | D(3, 2) | D(4, 3) | D(3, 2) |
| emulsifier | Dodecane | (μm) | (μm) | (μm) | (μm) |
| concentration | concentration | 1 day | 1 day | 3 weeks | 3 weeks |
| (% weight) | (% weight) | storage | storage | storage | storage |
| 0.25 (0.25) | 2 | 45.91 | 39.01 | 45.12 | 38.36 |
| 0.24 (0.25) | 6 | 113.69 | 105.56 | 106.92 | 94.57 |
| 0.23 (0.25) | 10 | 141.06 | 127.12 | 140.60 | 122.56 |
| 0.49 (0.5) | 2 | 53.33 | 34.23 | 53.19 | 36.21 |
| 0.47 (0.5) | 6 | 67.14 | 58.81 | 61.84 | 53.96 |
| 0.45 (0.5) | 10 | 73.50 | 64.33 | 64.81 | 55.27 |

### Example 4

### Preparation of Dodecane-in-Water Emulsions Stabilised with 0.5% W/W Agar Gelled Particle Emulsifier

The gelled particle emulsifier of example 1 was collected from the storage solution by centrifugation for 10 minutes at 3000 rpm in a bench centrifuge (MSE Centaur 2 centrifuge). The supernatant was discarded and 0.5% w/w gelled particle emulsifier was re-dispersed in 0.1% w/w aqueous citric acid using a Silverson LR4 high shear mixer set at high speed for 3 minutes. To this dispersion was added either 2%, 10% or 20% w/w dodecane. Emulsions were then prepared using a Silverson L4R high shear mixer set at high speed (speed setting 5) for 2 minutes.

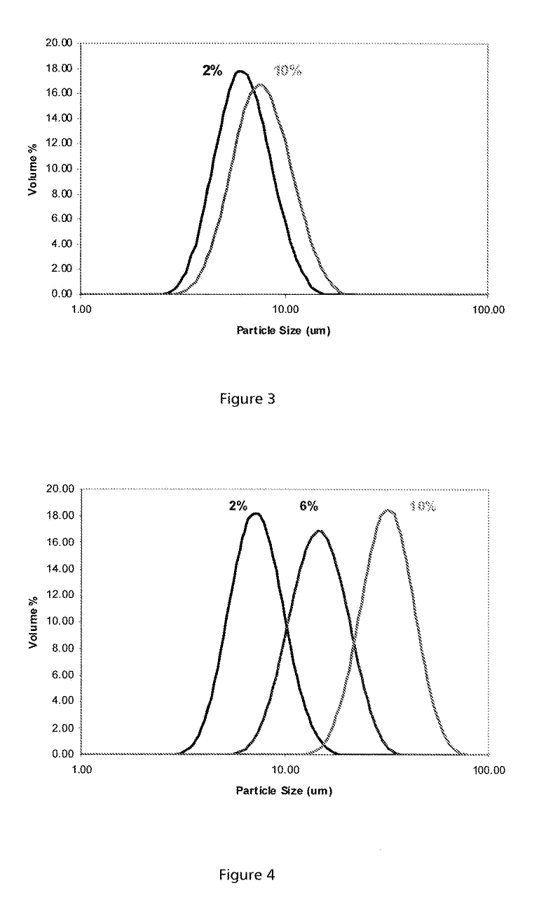

FIG. 3 shows the oil droplet size measured on a Malvern Mastersizer 2000. The method was the same as that used in example 1, except the refractive index of the dispersed particles (dodecane) was set to 1.421.

### Example 5

### Preparation of Dodecane-in-Water Emulsions Stabilised with 0.25% W/W Agar Gelled Particle Emulsifier

Emulsions were prepared using the same method as that described for example 4, except 0.25% w/w agar gelled particle emulsifier was used and the dodecane levels tested were 1%, 2%, 6% and 10% w/w.

FIG. 4 shows the oil droplet size, measured using the Malvern Mastersizer 2000 using the method described in example 4

### Example 6

### Preparation of Dodecane-in-Water Emulsions Stabilised with 0.1% W/W Agar Gelled Particle Emulsifier

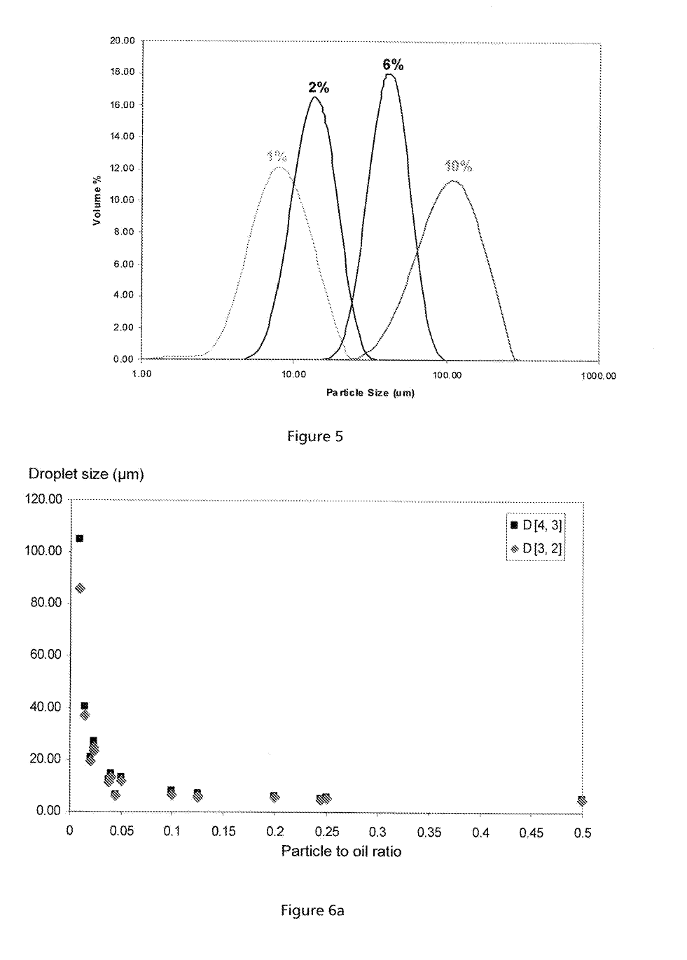

Emulsions were prepared using the same method as that described for example 4, except 0.1% w/w agar gelled particle emulsifier was used and the dodecane levels tested were 0.5%, 1%, 2%, 6% and 10% w/w FIG. 5 shows the oil droplet size, measured using the Malvern Mastersizer 2000 using the method described in example 3.

Table 2 summarises the agar gelled particle emulsifier stabilised emulsion oil droplet size data for the dodecane-in-water emulsions of examples 4, 5 and 6. The droplet sizes quoted are the mean volume weighted drop size D(4,3) and the mean surface area weighted or Sauter mean drop size, D(3,2). The agar to dodecane weight ratio is based on an agar concentration in the gelled particle emulsifier form of 8% w/w (see example 1). The particle concentrations quoted in parentheses are the particle concentrations in the aqueous phase

TABLE 2
Agar gelled particle emulsifier stabilised emulsion oil droplet size
data for the dodecane-in-water emulsions of examples 4, 5 and 6

| Agar gelled | | Agar gelled | | | |
|---|---|---|---|---|---|
| particle emulsifier | Dodecane | particle emulsifier | Agar to | | |
| concentration | concentration | to dodecane | dodecane | D(4, 3) | D(3, 2) |
| (% weight) | (% weight) | weight ratio | weight ratio | (μm) | (μm) |
| 0.10 (0.1) | 0.5 | 0.200 | 0.0160 | 6.16 | 5.65 |
| 0.10 (0.1) | 1 | 0.100 | 0.0080 | 8.21 | 6.78 |
| 0.10 (0.1) | 2 | 0.050 | 0.0040 | 13.17 | 11.92 |
| 0.09 (0.1) | 6 | 0.015 | 0.0012 | 40.31 | 37.06 |
| 0.09 (0.1) | 10 | 0.009 | 0.0007 | 104.9 | 85.49 |
| 0.25 (0.25) | 0.5 | 0.500 | 0.0400 | 5.34 | 4.89 |
| 0.25 (0.25) | 1 | 0.250 | 0.0200 | 5.86 | 5.39 |
| 0.25 (0.25) | 2 | 0.125 | 0.0100 | 6.19 | 5.68 |
| 0.25 (0.25) | 2 | 0.125 | 0.0100 | 7.05 | 6.21 |
| 0.24 (0.25) | 6 | 0.040 | 0.0031 | 12.47 | 11.33 |
| 0.24 (0.25) | 6 | 0.040 | 0.0031 | 14.73 | 13.36 |
| 0.23 (0.25) | 10 | 0.023 | 0.0018 | 27.00 | 24.92 |
| 0.23 (0.25) | 10 | 0.023 | 0.0018 | 25.20 | 23.29 |
| 0.49 (0.5) | 2 | 0.245 | 0.0196 | 5.34 | 4.89 |
| 0.45 (0.5) | 10 | 0.045 | 0.0036 | 6.64 | 6.00 |
| 0.40 (0.5) | 20 | 0.020 | 0.0016 | 21.07 | 19.23 |

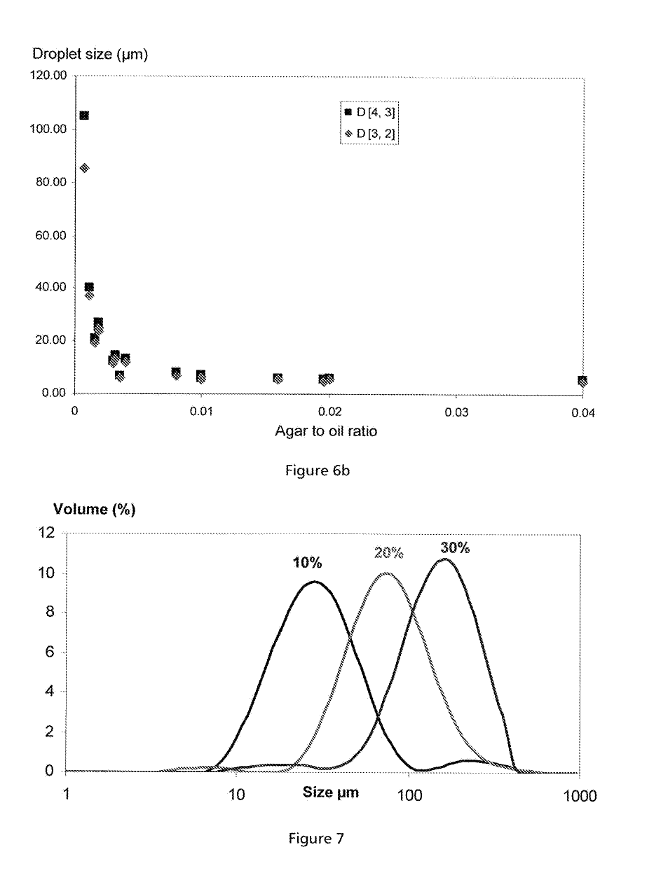

Table 2 indicates how little agar, in gelled particle emulsifier form, is needed to stabilise reasonably high levels of dodecane in a dodecane-in-water emulsion. FIG. 6 shows the variation of D(4,3) and D(3,2) with particle oil ratio.

### Example 7

### Preparation of Silicon Oil-in-Water Emulsions Stabilised with Agar Gelled Particle Emulsifier

A new batch of particles was prepared using the method described in example 1. The particle size was determined using a Malvern Mastersizer 2000 by the method described in example 1. The volume weighted particle size D(4,3) and the mean surface area weighted or Sauter mean particle size D(3,2). were 0.96 μm and 0.38 μm respectively. The water content of the particles was 93%

Emulsions were prepared using the same method as that described for example 3. The oil used in this example was a silicon oil (DC 200® fluid 50CST) supplied by Dow Corning. 4 emulsions were prepared: 30% silicon oil stabilised by 0.5% w/w agar particles, 20% w/w silicon oil with 0.5% w/w particles and 10% w/w silicon oil with 0.5% w/w particles. Table 3 shows the oil droplet size data measured on a Malvern Mastersizer 2000 for the emulsions. The method was the same as that used in example 1, except the refractive index of the dispersed particles (silicon oil) was set to 1.400 The droplet sizes quoted are the mean volume weighted drop size D(4,3) and the mean surface area weighted or Sauter mean drop size, D(3,2). Also shown in table 3 are the oil droplet size data after 2 weeks storage at 5° C.

TABLE 3
Agar gelled particle emulsifier stabilised emulsion oil droplet size
data for the silicon oil-in-water emulsions of example 7

| Agar gelled | | D(4, 3) | D(3, 2) | D(4, 3) | D(3, 2) |
|---|---|---|---|---|---|
| particle emulsifier | Silicon oil | (μm) | (μm) | (μm) | (μm) |
| concentration (% | concentration | 1 day | 1 day | 2 weeks | 2 weeks |
| weight) | (% weight) | storage | storage | storage | storage |
| 0.45 (0.50) | 10 | 37.52 | 26.75 | 42.97 | 24.42 |
| 0.40 (0.25) | 20 | 82.94 | 55.78 | 101.43 | 65.67 |
| 0.35 (0.5) | 30 | 152.28 | 133.43 | 182.89 | 128.41 |

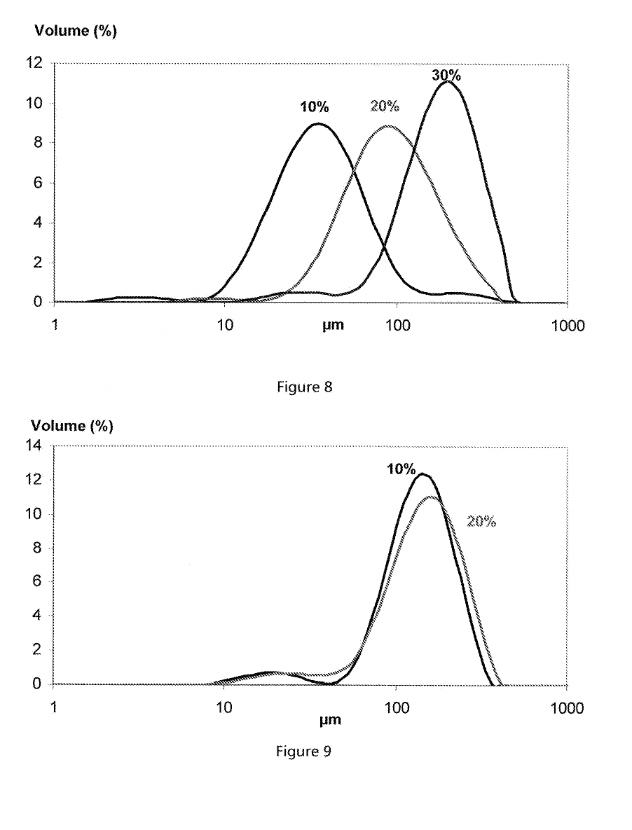

FIG. 7 shows the oil droplet size distribution of silicon oil-in-water emulsions stabilised by 0.5% w/w agar gelled particle emulsifier according to example 7 obtained by light scattering for 10%, 20% and 30% w/w silicon oil. FIG. 8 shows the data form the same emulsions after 2 weeks storage at chill temperature.

Comparison of FIGS. 7 and 8 suggests good stability of the emulsions with no large amounts coalescence.

### Example 8

### Preparation of Medium Chain Length Triglyceride Oil (MCT) Oil-in-Water Emulsions Stabilised with Agar Gelled Particle Emulsifier

Emulsions were prepared using the same method and particles as in example 7, except that the particles were dispersed in 0.1% w/w citric acid prior to preparing an emulsion. The MCT oil used was supplied by Danisco (Grinsted® MCT 60X/C).

Table 4 shows the oil droplet size date measured on a Malvern Mastersizer 2000 for the emulsions. The method was the same as that used in example 1, except the refractive index of the dispersed particles (MCT oil) was set to 1.449 The droplet sizes quoted are the mean volume weighted drop size D(4,3) and the mean surface area weighted or Sauter mean drop size, D(3,2). Also shown in table 4 are the oil droplet size data after 2 weeks storage at 5° C.

TABLE 4
Agar gelled particle emulsifier stabilised emulsion oil droplet
size data for the MCT oil-in-water emulsions of example 8

| Agar gelled | | D(4, 3) | D(3, 2) | D(4, 3) | D(3, 2) |
|---|---|---|---|---|---|
| particle emulsifier | MCT oil | (μm) | (μm) | (μm) | (μm) |
| concentration | concentration | 1 day | 1 day | 2 weeks | 2 weeks |
| (% weight) | (% weight) | storage | storage | storage | storage |
| 0.45 (0.50) | 10 | 135.06 | 53.52 | 138.61 | 45.77 |
| 0.40 (0.25) | 20 | 145.35 | 99.89 | 147.08 | 92.49 |

FIG. 9 shows the oil droplet size distribution of the MCT oil-in-water emulsions stabilised by 0.5% w/w agar gelled particle emulsifier according to example 8 obtained by light scattering for 10% and 20% w/w MCT oil.

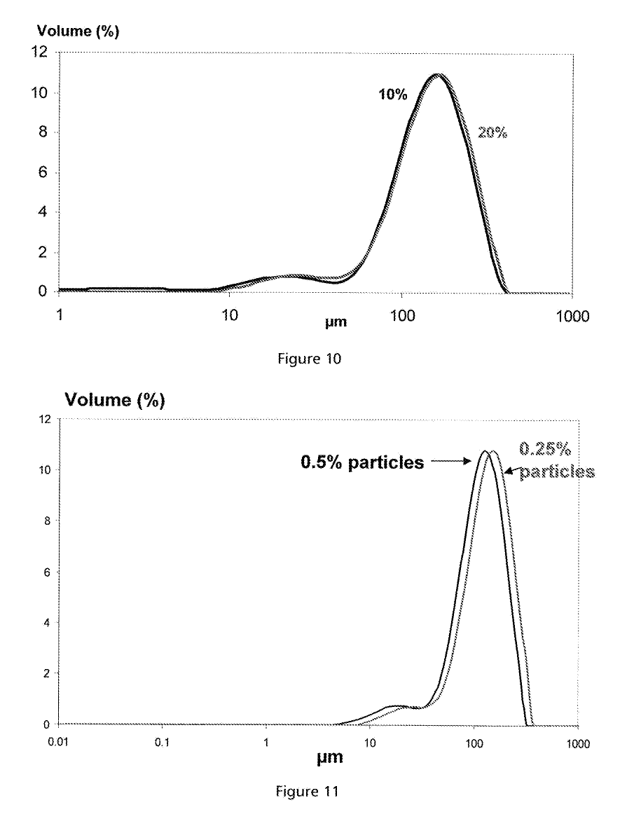

FIG. 10 show the data from the same emulsions after 2 weeks storage at chill temperature. Comparison of FIGS. 9 and 10 suggests very good stability of the emulsions, with no coalescence observed.

### Example 9

### Preparation of a Soya Bean Oil-in-Water Emulsion Stabilised with Agar Gelled Particles

Emulsions were prepared using the same method and particles as in example 7, except that the particles were dispersed in 0.5% w/w citric acid prior to preparing an emulsion. The soya bean oil was supplied by Sigma (Code: S 738). The particle concentrations employed was 0.25% w/w The soya beam oil level used was 10% w/w

FIG. 11 shows the oil droplet size distribution of the soya bean oil oil-in-water emulsions stabilised by 0.25% w/w agar gelled particle emulsifier according example 9 obtained by light scattering for 10% w/w soya bean oil emulsions.

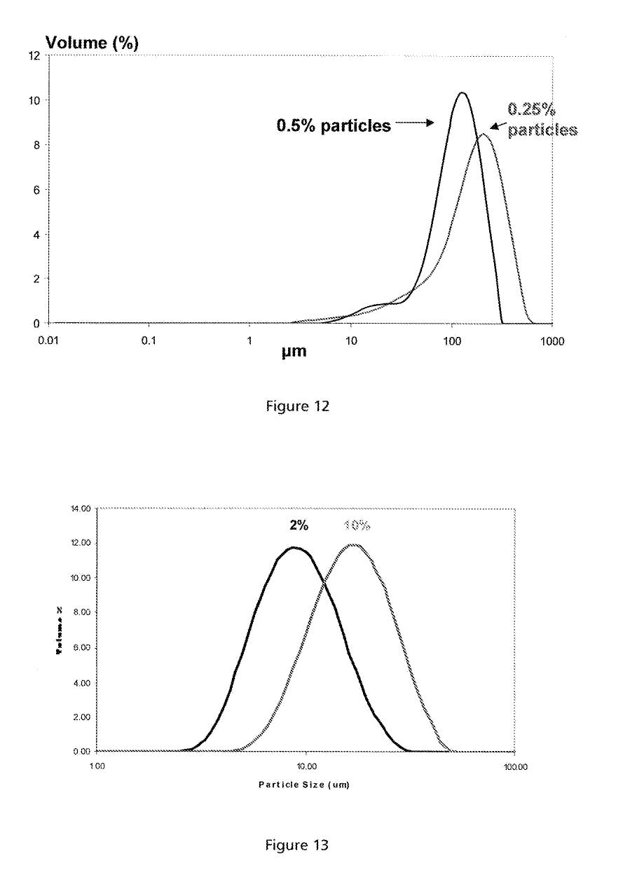

FIG. 12 shows the oil droplet size distribution of soya bean oil oil-in-water emulsions stabilised by 0.25% w/w agar gelled particle emulsifier according to example 9 obtained by light scattering for 10% w/w soya bean oil after 1 week storage at chill temperature.

Comparison of FIGS. 11 and 12 show good stability for the 10% w/w soya bean oil emulsion stabilised with 0.25% w/w agar particles.

### Example 10

### Preparation of Dodecane-in-Water Emulsions Stabilised with Pectin Gelled Particle Emulsifier

Dodecane-in-water emulsions were prepared as described in examples 4, 5 and 6 but with pectin gelled particle emulsifier rather than agar gelled particle emulsifier. The emulsion prepared with 0.5% w/w pectin gelled particle emulsifier of example 4 and 20% w/w dodecane was replaced with one with 6% w/w dodecane.

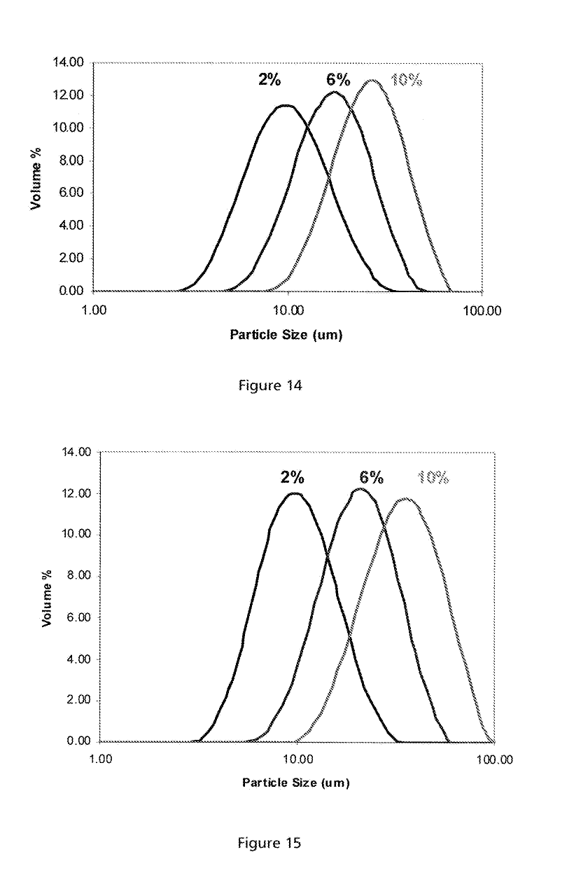

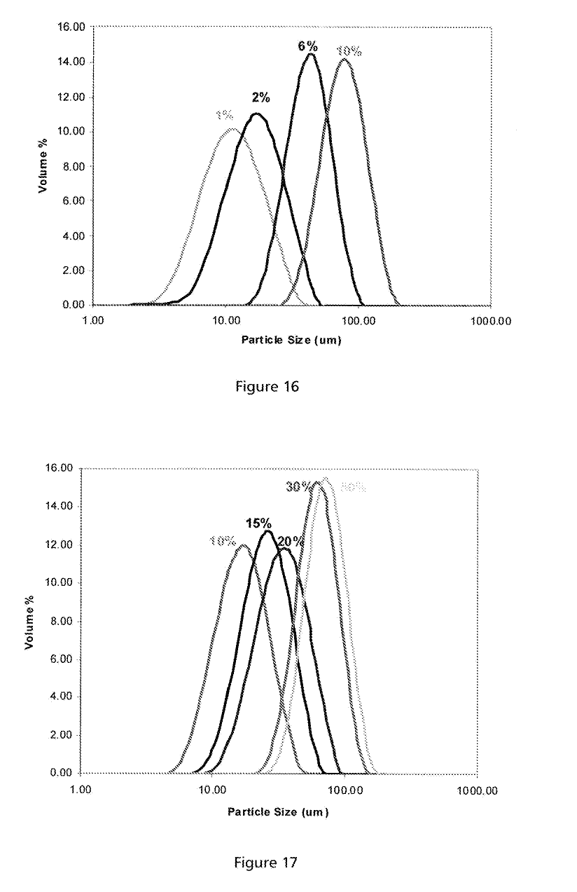

FIGS. 13 14 and 16 show the oil droplet size, measured using the Malvern Mastersizer 2000 using the method described in example 3. FIG. 16 shows the droplet size of the dodecane-in-water emulsions of FIG. 14 after 1 week of storage at chill temperature. The droplet size remained the same.

### Example 11

### Preparation of Dodecane-in-Water Emulsions Stabilised with 0.5% W/W Pectin Gelled Particle Emulsifier and with Higher Levels of Dodecane

Emulsions were prepared using the same method as that described for example 10, except that the dodecane levels tested were 10%, 15%, 20%, 30%, 40% and 50% w/w.

FIG. 17 shows the oil droplet size, measured using the Malvern Mastersizer 2000 using the method described in example 3.

Table 5 summarises the pectin gelled particle emulsifier stabilised emulsion oil droplet size data for the dodecane-in-water emulsions of examples 9 and 10 in a similar manner to as shown in table 2. The pectin to dodecane weight ratio is based on a pectin concentration in the gelled particle emulsifier form of 7% w/w (see example 2).

TABLE 5
Pectin gelled particle emulsifier stabilised emulsion oil droplet size
data for the dodecane-in-water emulsions of examples 9 and 10.

| Pectin gelled | | Pectin gelled | | | |
|---|---|---|---|---|---|
| particle | | particle | | | |
| emulsifier | Dodecane | emulsifier to | Pectin to | | |
| concentration | concentration | dodecane | dodecane | | |
| (% weight) | (% weight) | weight ratio | weight ratio | D(4, 3) | D(3, 2) |
| 0.1 (0.1) | 0.5 | 0.200 | 0.0140 | 8.87 | 7.21 |
| 0.1 (0.1) | 1 | 0.100 | 0.0070 | 11.71 | 9.13 |
| 0.1 (0.1) | 2 | 0.050 | 0.0035 | 17.07 | 13.62 |
| 0.09 (0.1) | 6 | 0.015 | 0.0011 | 42.11 | 37.09 |
| 0.09 (0.1) | 10 | 0.009 | 0.0006 | 76.84 | 67.41 |
| 0.25 (0.25) | 0.5 | 0.500 | 0.0035 | 7.98 | 6.67 |
| 0.25 (0.25) | 1 | 0.250 | 0.0175 | 8.41 | 7.02 |
| 0.25 (0.25) | 2 | 0.125 | 0.0088 | 10.21 | 8.37 |
| 0.24 (0.25) | 6 | 0.040 | 0.0028 | 17.10 | 14.33 |
| 0.23 (0.25) | 10 | 0.023 | 0.0016 | 33.53 | 27.96 |
| 0.49 (0.5) | 2 | 0.245 | 0.0172 | 9.31 | 7.74 |
| 0.47 (0.5) | 6 | 0.078 | 0.0055 | 13.50 | 10.68 |
| 0.45 (0.5) | 10 | 0.045 | 0.0032 | 16.79 | 14 |
| 0.43 (0.5) | 15 | 0.028 | 0.0020 | 25.40 | 21.59 |
| 0.40 (0.5) | 20 | 0.020 | 0.0014 | 33.91 | 28.22 |
| 0.35 (0.5) | 30 | 0.012 | 0.0008 | 60.29 | 53.82 |
| 0.30 (0.5) | 40 | 0.008 | 0.0005 | 68.35 | 59.62 |
| 0.25 (0.5) | 50 | 0.005 | 0.0004 | 69.92 | 62.63 |

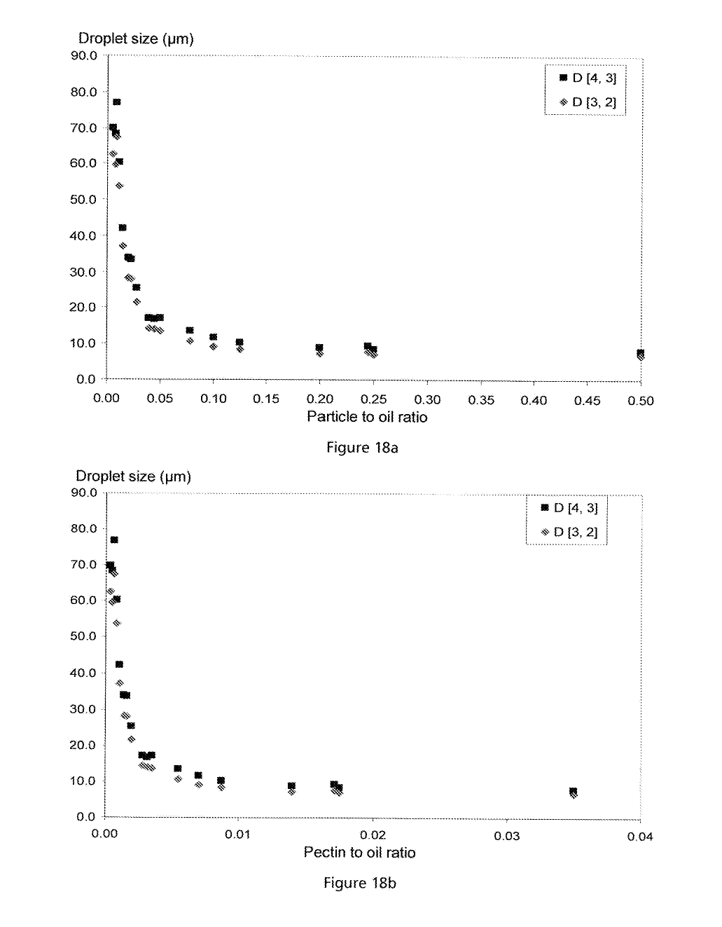

Inspection of the data in table 5 indicates how little pectin, in gelled particle emulsifier form, is needed to stabilise reasonably high levels of dodecane in a dodecane-in-water emulsion. FIG. 18a shows the variation of D(4,3) and D(3,2) with particle concentration and FIG. 18b show the variation D(4,3) and D(3,2) with the total amount of pectin used.

1. An oil-in-water emulsion comprising 0.001-50%, preferably 0.001-30%, more preferably 0.001-10% w/w oil and 0.001 to less than 0.5%, preferably 0.001-0.4%, more preferably 0.01-0.4% w/w a gelled particle emulsifier, wherein the gelled particle emulsifier comprises at least one gellable hydrophilic polysaccharide, wherein particles of the gelled particle emulsifier have a largest dimension of 3-1000 nm, preferably 5-500 nm, more preferably 10-200 nm.

2. An emulsion according to claim 1, wherein the weight ratio of gelled particle emulsifier to oil is 0.001-0.5:1, preferably 0.005-0.25:1, more preferably 0.01-0.1:1.

3. An emulsion according to claim 1, wherein the gelled particle emulsifier has an aspect ratio in the range of 1:1 to 10:1.

4. An emulsion according to claim 1, containing less than 0.1% w/w of hydrophilic polysaccharide.

5. An emulsion according to claim 1, wherein the gellable polysaccharide is selected from the group consisting of agar, agarose, gellan and pectin.

6. An emulsion according to claim 1 comprising no further emulsifier.

7. An emulsion according to claim 1 in the form of a food product or a home care product or a personal care product or a pharmaceutical product.
